(19) United States
(12) Patent Application Publication
(10) Pub. No.: US 2015/0257968 A1
(43) Pub. Date: Sep. 17, 2015

(54) SOFT CONFORMAL COMPRESSION DEVICES AND METHODS

(71) Applicants: Carl E. VAUSE, Concord, MA (US); Joshua Aaron Lessing, Cambridge, MA (US); Ryan Richard Knopf, Cambridge, MA (US); Mitchell Ross Zakin, Andover, MA (US)

(72) Inventors: Carl E. VAUSE, Concord, MA (US); Joshua Aaron Lessing, Cambridge, MA (US); Ryan Richard Knopf, Cambridge, MA (US); Mitchell Ross Zakin, Andover, MA (US)

(21) Appl. No.: 14/644,910

(22) Filed: Mar. 11, 2015

Related U.S. Application Data
(60) Provisional application No. 61/950,961, filed on Mar. 11, 2014.

(51) Int. Cl. A61H 9/00; A61F 13/00

(52) U.S. Cl. CPC A61H 9/0007; A61F 13/00059; A61F 13/00068; A61F 2013/0028; A61F 2013/00127; A61F 2013/00174

(57) ABSTRACT

A soft conformal compression device for treatment of a wound on a patient's limb, the device including a network of pneumatic actuators and a wound dressing. The pneumatic actuators may be activated by applying a source of a gas to an inlet on the device, which may cause the device to curve, bend, or otherwise deform in a predetermined manner to encircle a patient's limb and urge the wound dressing toward the wound.

### FIELD OF THE DISCLOSURE

This application relates to medical devices for treating patients. More particularly, the application relates to soft-robotic devices for wound management.

### BACKGROUND OF THE DISCLOSURE

Generally, bandages are soft materials that are applied to the exterior of the body to provide mechanical support, absorb drained fluids, or act as a barrier between a portion of the body such as a wound and the external environment. Bandages are typically formed from woven textiles, and may include a wide variety of additives such as antiseptics (e.g. polyhexamethylene biguanide), drugs for transdermal delivery (e.g. nicotine, scopolamine, estrogen, etc.), coatings or membranes to prevent adhesion to wounds (e.g. mylar membranes), and/or adhesives to keep the bandage in contact with the skin. Bandages are formed as strips, tubes, sheets, and any other shapes which are suitable for their intended uses. They are typically flexible (capable of being deformed) and/or elastic (tending to return to their unstressed-configurations after being deformed), and are typically deployed so as to apply pressure to at least a portion of the body being treated. Bandages may provide varying degrees of pressure depending on their sizing and elasticity relative to the size of the patient and the area being treated. For some applications, such as the treatment of topical abrasions or lacerations, relatively little pressure is needed. For others, such as the treatment of hemorrhage or orthopedic conditions, more pressure may be called for.

Although the simplicity of currently-used bandages offers certain advantages such as low cost and ease of use, it also constrains their efficacy: existing bandages are most useful in settings where the need for support, absorption, or prevention of infiltration does not change over time. For treatment of conditions that do change over time or that require complex applications of force, a single bandage is generally inadequate, and multiple bandages may be required over time. In addition, bandages that apply substantial pressure may need to be removed in order to ensure that tissues adjacent to the region being treated receive sufficient blood perfusion. For traumatic degloving wounds, burns, compound fractures and similar injuries, traditional bandages may simply be inadequate, particularly if these injuries occur in places like the battlefield in which complex medical care is not readily available. Traditional bandages attach only to the surface of the wound and are not necessarily conformal to its margins, particularly if they are irregular, potentially allowing the loss of blood and other fluids as well as failing to completely prevent external contaminates and infectious particles from entering the wound. These shortcomings may be compounded further if the patient cannot be moved enough to dress the entire wound.

Recent advances in the field of “soft” robotic manipulators, or “soft robots,” have the potential to improve bandage design generally and wound care in particular. (See, for instance, International Patent Application Pub. No. WO2012/148472 by Ilievsky et al. entitled “SOFT ROBOTIC ACUATORS”, the entire disclosure of which is incorporated by reference herein for all purposes). Soft robots comprising elastomeric or extensible bodies and/or flexible polymeric or textile actuators rather than hard plastic or metal ones are remarkably well suited to the manipulation of delicate objects such as injured human tissue. However, in spite of their advantages over existing technologies, soft robots have not yet found widespread use in medicine.

The present invention, in its various embodiments, applies soft robotic technology to address the shortcomings of traditional bandages.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

An exemplary embodiment of a soft conformal compression medical device for treatment of a wound on a patient's limb in accordance with the present disclosure may include a network of pneumatic actuators and a wound dressing. The pneumatic actuators may be activated by providing a source of a gas to an inlet on the device, which may cause the device to curve, bend, or otherwise deform in a predetermined manner to encircle a patient's limb and urge the wound dressing toward the wound.

A method of treating a wound in accordance with the present disclosure may include placing, adjacent a wound on a portion of a patient's body, a conformal compressive device, the device configured to at least partially encircle the portion of the patient's body and including a wound dressing and a network of pneumatic actuators configured to urge the wound dressing toward the wound when activated, and activating the network of pneumatic actuators.

### BRIEF DESCRIPTION OF THE DRAWINGS

Aspects of the invention are described below with reference to the following drawings in which like numerals reference like elements, and wherein:

FIG. 1A is a perspective view illustrating a conformal compression device according to certain embodiments of the invention;

FIG. 1B is a perspective, partially disassembled view illustrating the conformal compression device shown in FIG. 1A;

FIG. 2A is an isometric bottom view illustrating a conformal compression device according to certain embodiments of the invention;

FIG. 2B is an isometric top view illustrating the conformal compression device shown in FIG. 2A;

FIG. 3A-D are a series of schematic views illustrating several steps in the use of the conformal compression device shown in FIG. 2A to close a wound;

FIG. 4 is a cross-sectional view illustrating the conformal compression device shown in FIG. 2A;

FIG. 5A is a top view illustrating a conformal compression device according to certain embodiments of the invention;

FIG. 5B is a bottom view illustrating the conformal compression device shown in FIG. 5A;

FIG. 6A is a schematic view illustrating a conformal compression device according to certain embodiments of the invention in a non-actuated state;

FIG. 6B is a schematic view illustrating the conformal compression device shown in FIG. 6A in an actuated state.

Unless otherwise provided in the following specification, the drawings are not necessarily to scale, with emphasis being placed on illustration of the principles of the invention.

### DETAILED DESCRIPTION

Soft robotic devices for treating wounds and applying pressure to limbs are described herein. The devices preferably comprise networks of pneumatic actuators referred to herein and elsewhere as “Pneu-Nets.” Individual pneumatic actuators within such networks can function in several ways. One category of actuators is flexible and/or compliant at ambient pressure (e.g. 1 atm), becoming rigid when externally pressurized or placed under vacuum internally. These actuators include paired elastomeric beams comprising opposing toothed patterns separated by an open-cell foam. Under vacuum, the dead air within the foam is evacuated, drawing the elastomeric beams together such that the toothed patterns interdigitate, forming a single, relatively rigid beam.

A second type of actuator, described by Ilievsky et al., is substantially linear and/or has a first curvature at a ambient pressure, but becomes (more) curved upon the application of internal pressure or vacuum. Actuators of this type generally comprise a plurality of chambers arranged in an array. The chambers are defined by a plurality of walls, and the various configurations of the actuators are determined in part by the relative flexibility and/or elasticity of the walls. Specifically, walls that are more elastic (e.g. thinner elastomeric walls) will tend to elongate and/or bend as the internal pressure of the chamber increases, while walls that less elastic (e.g. thicker elastomeric walls) will not bend or elongate to the same degree. In addition, some walls can be flexible but not elastic such that, when the internal pressure of the chamber increases, the wall bends without elongating. In some embodiments, the chambers in the array are separated from one another by relatively thick walls. The chambers in the array are further defined by a first side that is covered by a relatively thin elastic membrane capable of expanding in length as the pressure within the chamber increases and a second side, that does not vary significantly in length as the internal pressure of the chamber increases or decreases (referred to by Ilievsky et al. as a “strain-limiting” portion of the chamber). As the pressure increases, the first side expands along with the internal volume of the chamber, thereby causing the chamber to curve about the second side wall. Alternatively or additionally, if the internal pressure within the chamber is reduced below the ambient pressure, the first side wall will buckle, permitting the chamber to curve away from the second side wall. The ultimate shape and action of the device can be tuned, among other ways, by adjusting the relative thicknesses and elasticities of the various walls defining the chambers, and/or by varying these thicknesses and/or elasticities across the array.

More generally, pneumatic elements used in the invention respond to increases in internal pressure by expanding, particularly in the regions that are most compliant or have the lowest stiffness. In devices with homogeneous elastomer structures, the regions that expand the most are those with the thinnest walls (which are the structures with the lowest resistance to stretching). Pressurization and expansion in these regions further thins the walls, and increases the volume of the channel. To accommodate the asymmetric elongation of two opposite walls of the channel, the structure surrounding the expanding volume bends. Upon pressurization, a single channel spanning the length of a rectangular slab causes the slab to bend around the axis of the channel. Multiple channels have an additive effect; upon pressurization, an actuated network of channels can generate complex shapes in elastomeric structures. The choice of materials, coupled with the design of the channels and the speed of inflation, determines the response of the device to applied pressure. The pressure necessary to achieve a particular amplitude of actuation scales with the stiffness of the materials and with the ratio of elastomeric polymer material in the chamber relative to the volume of the chamber.

Passive and active loading capacity of actuators (the maximum sustainable load) correlates to the stiffness of the materials used for fabrication—stronger materials are able to support larger loads, but typically cannot sustain very large strains. Agility—the ability to create intricate movements, and to do so rapidly—requires bending to small radii of curvature, and is thus easier to achieve in materials able to sustain high strains. Composite structures, in which materials with different stiffnesses join to form a channel, are useful for programming the directionality of actuation, and provide properties that benefit from the combined mechanical properties of the different materials. As with single-material devices, the composite channels expand upon pressurization at the most compliant region. Since the different compliances are now not controlled by the channel design, but largely determined by the choice of materials and their layout, regions made from a more elastic material will expand.

Stacking or connecting the components described above creates structures that provide complex motion and require only a single source of pressure. Appropriate distribution, configuration, and size of the Pneu-Net elements determine the resulting movement. Pneumatic and/or hydraulic systems are attractive for medical applications, particularly those performed outside of the clinic, because air has low viscosity, and permits rapid actuation; since air is compressible, it is easy to store, light and environmentally benign; biocompatible hydraulic fluids such as saline are also easy to store and are environmentally benign.

Devices comprising Pneu-Nets can generate a wide variety of movements including gripping, inversion of shape from convex to concave, and undulating shape changes. Additionally, their modular structure allows designers the freedom to design each layer or segment of a Pneu-Net device separately, tailoring the structure of each part to achieve a particular function, and then join them together. For example, a ridged texture may be added to a skin-contacting layer in order to enhance grip. The textured surface is more compliant than a solid surface of the same material and provides more traction. In some embodiments, the textured surface is achieved through the actuation of Pneu-Net actuators: at atmospheric pressure, the surface, and the actuation of the Pneu-Net results in the formation of corrugations, striations, or other surface textures.

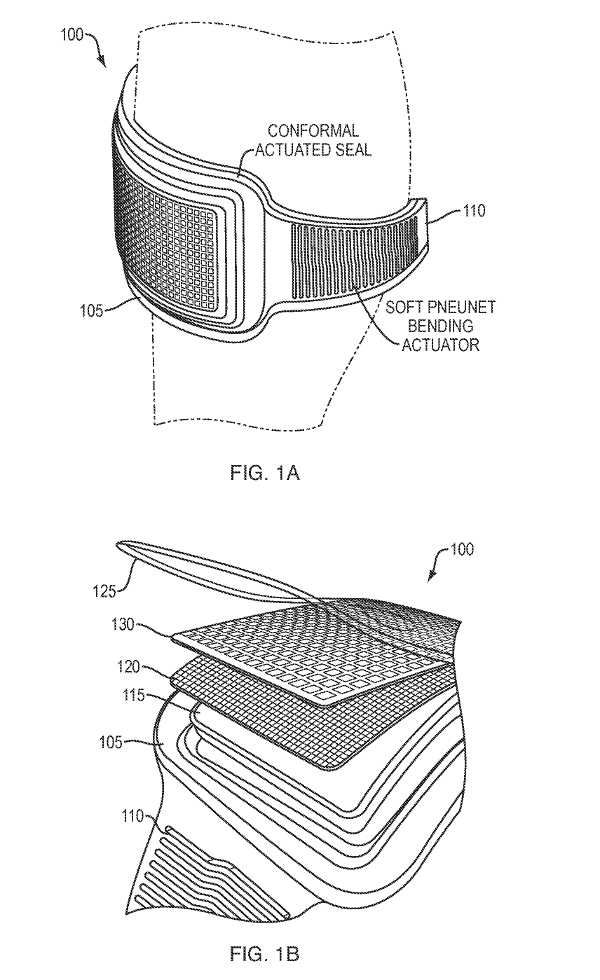

Referring now to FIG. 1A, an exemplary soft conformal cover (CC) 100 according to certain embodiments the present invention is adapted to treat wounds on the limbs (i.e. the arm and/or the leg, though similar devices may be used to treat the head, neck, shoulder or any other body part which can be at least partially encircled by CC 100). The CC 100 includes a first portion 105 for placement over a wound and a second portion 110 that at least partially encircles the limb in order to urge the first portion 105 toward the wound being treated. When in contact with the limb, the first portion 105 generally forms a conformal seal around the wound, providing a barrier to environmental elements while applying pressure to the wound being treated. The seal is provided, in various embodiments, by the use in the first portion 105 optionally of at least one Pneu-Net actuator which, when actuated, urges the edges of the first portion 105 toward the skin surrounding the wound. Alternatively or additionally, an inflatable gasket, or simply an elastomeric and/or adhesive material may be disposed on a surface of the CC 100 in order to help form a conformal seal around the wound. The first portion 105 is, in various alternative embodiments, sized and shaped to apply pressure to the wound without sealing the wound, or conformally seal the wound without applying direct pressure to the wound.

The first portion 105, as shown in FIG. 1B, includes an innermost wound dressing layer 115, for instance a gauze or hydrogel layer, a water-permeable layer 120 to permit fluids generated by the wound to diffuse away from the wound itself, and an external barrier layer 125 which prevents infiltration of the wound by external matter. The first portion 105 also optionally includes a sensor layer 130 comprising one or more sensors for detecting, for instance, molecules emitted from the wound that are useful in assaying wound healing. The sensor layer 130 shown in FIGS. 1A-B, for instance, includes a rectangular array of O₂ sensors coupled to display elements 131 such as light-emitting diodes (LEDs) that may display indicia indicating the state of the wound, such as by signaling relative levels of O₂ emissions across the array. In another embodiment, the sensor layer 130 is adapted to provide colorimetric detection of bacteria, or of clotting factors, etc.

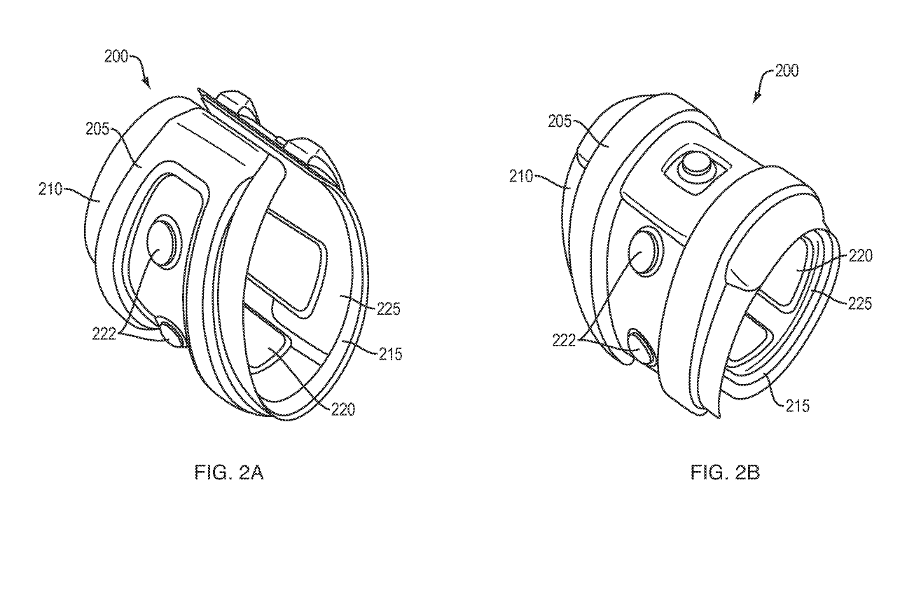

Turning to FIGS. 2-4, in another embodiment of the present disclosure a CC 200 has a unibody construction comprising five layers:
An external layer 205 comprising a soft, conformal, and rugged layer capable of resisting puncture (e.g. a thermoplastic urethane, neoprene or other suitable material);
An elastomeric actuation layer 210 comprising the Pneu-Net, a plurality of rapid pneumatic actuators which enable the CC 200 to encircle and enclose a wounded limb, conforming the dressing to the wound topography and securing the device in place;
A gasket layer 215 comprising an adhesive and preferably strain-limiting pneumatic gasket to seal the wound from external contaminants, prevent the loss of fluid, and permitting irrigation and/or suction of the wound. In addition, in certain embodiments, the gasket layer 215 facilitates the application of fluid therapeutic, such antibiotics, which are optionally pre-mixed with an irrigant or which are contained in a chamber formed within the gasket layer 215. The gasket layer 215 includes a plurality of fluid channels or paths 219 terminating in at least one port 216 connectable to an external fluid handling device (e.g. a syringe). In one non-limiting example, the port 216 may be a needle-penetrable, resealing septum or a needleless cap coupled to male or female luer-lock or slip-tip connectors (any suitable fluid connectors known in the art are suitable); such connectors are referred to generically as “catheter lock” or “hep-lock” connectors in the drawings;
A directed pressure layer 220, comprising separate directed pressure inflators 222 formed of Pneu-Net actuators adapted to provide directed pressure for treating specific acute vascular injuries or for fine-tuning or supplementing the pressures applied by the elastomeric actuation layer 210. For instance, the directed pressure layer 220 may include stiffening actuators as described above to facilitate the splinting of orthopedic injuries adjacent to the wound; and
A dressing layer 225 comprising specific wound treatment elements that are placed in close apposition with the wound itself. For instance, the dressing layer may include gauze or hydrogel, a perforated mylar coating to prevent sticking, as well as drugs or other bioactive agents for delivery to the wound (e.g. polyhexamethylene biguanide).

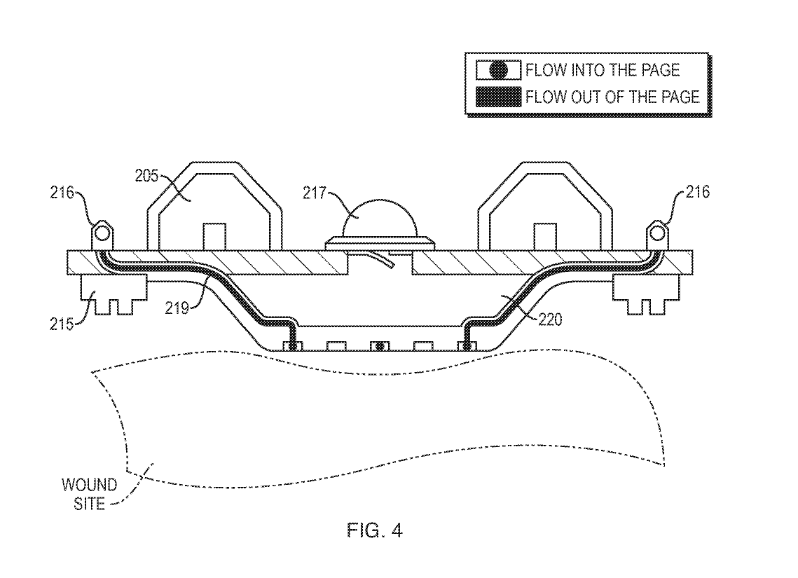

The CC 200 may further include an inflation mechanism 217, such as a manual inflation bulb (as shown in FIG. 4) or a compressed CO₂ cartridge, for inflating the actuation layer 210 and/or the directed pressure layer 220 with a gas. The inflation mechanism 217 may be separate from or integral to the CC 200.

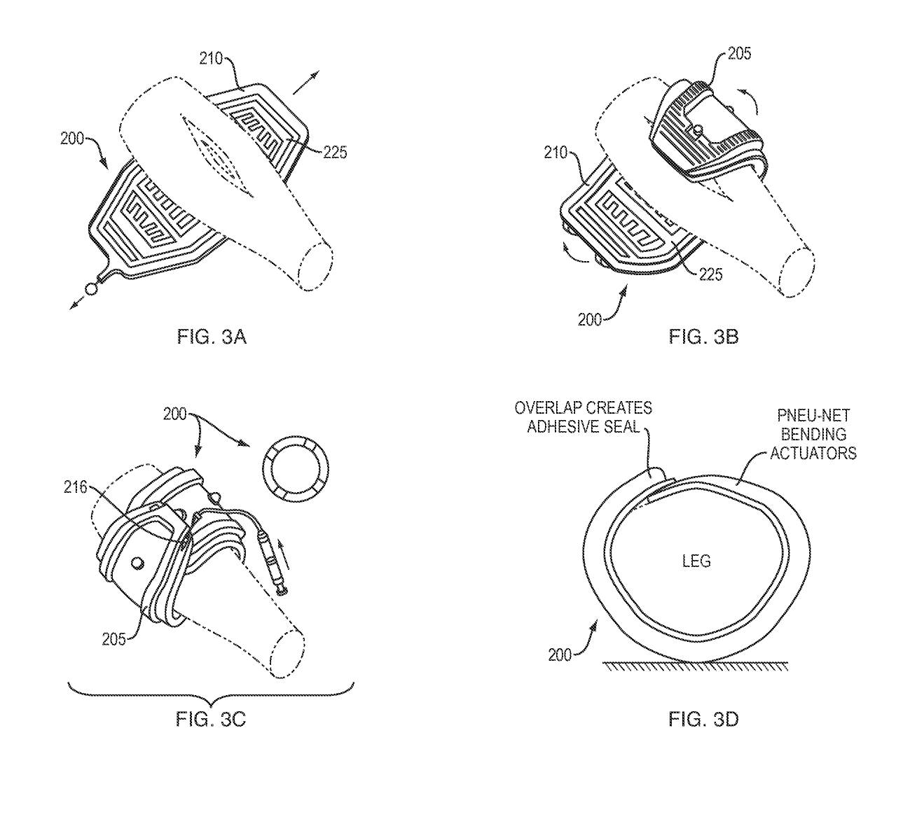

As shown in FIGS. 3A-3D, the CC 200 according to the invention may be placed beneath a wounded limb of a patient and then inflated. As the Pneu-Net of the CC 200 is inflated, actuators in the actuation layer 210 begin curving the CC 200 around the limb, bringing the dressing layer 225 into contact with the wound and applying pressure to the wound. As shown in FIG. 3C in inset, the relative volumes of individual chambers within the Pneu-Net of the actuation layer 210 may vary. For example, the chambers of the Pneu-Net overlying the wound may be inflated to greater volumes, thereby applying greater pressures to the wound then are applied elsewhere. Once deployed, a user optionally provides suction and/or irrigation to the wound via at least one of the ports 216 or, alternatively, suction and/or irrigant may be applied without user input, and a user may subsequently refill the irrigant supply and/or remove already-suctioned materials via the port or ports 216.

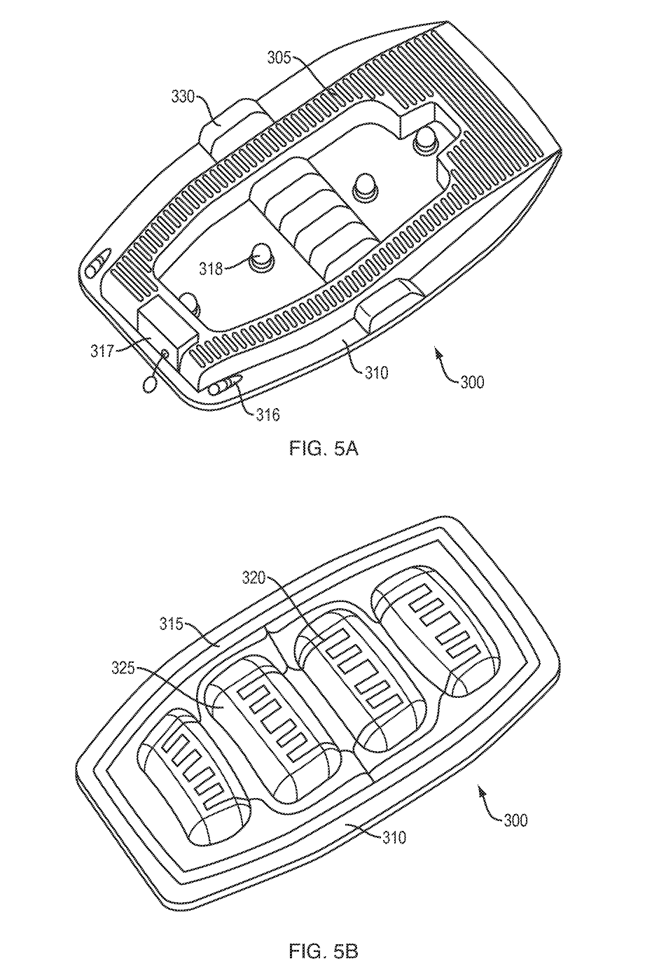

Turning to FIGS. 5A and 5B, in another embodiment of the present disclosure a CC 300 is illustrated. In addition to having an external layer 305, an actuation layer 310, a gasket layer 315, a directed pressure layer 320, a dressing layer 325, a port 316, and an inflation mechanism 317 similar to those of the CC 200 described above, the CC 300 may further include one or more static reinforcing members, such as a rigidizing spine 330, which may be formed of plastic, metal, composites, or other rigid materials and may provide the CC 300 with additional rigidity and structural support. The CC 300 may also include membrane inflators 318 disposed on an outer surface thereof, the actuation of which may provide pressure to directed pressure inflators 322 in the directed pressure layer 320 (as described above), which may in turn apply pressure to a wound. The directed pressure inflators 322 may be Pneu-Net actuators disposed over the wound-covering portion of the device as described above, but may also be simple balloons disposed on the inner surface of the CC 300. The directed pressure inflators 322 may include open face microfluidic channels or any other features described herein for providing therapy directly to a wound.

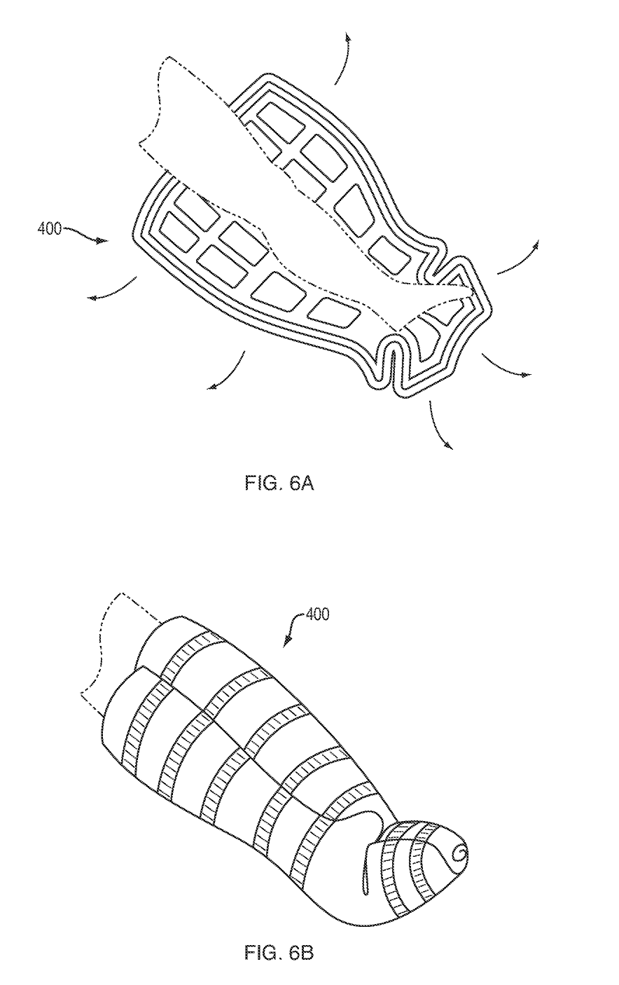

While the foregoing disclosure has focused on the treatment of wounds, CCs according to various embodiments of the invention can also be used to apply pressure or vacuum to limbs or other body parts that are not affected by specific, external wounds. In some embodiments, CCs are configured to provide heat and/or cold therapy to, for instance, injured musculoskeletal tissue. For instance, as shown in FIGS. 6A and 6B, in certain embodiments a CC 400 in accordance with the present disclosure, which may be substantially similar to the CC 200 and the CC 300 described above, can be used to substantially envelop and apply pressure to a limb such as a leg. Once deployed, the CC 400 can apply pressure and/or provide rigidity in order to stabilize the limb (e.g. functioning as a cast or a boot to protect a musculoskeletal injury), or it can provide oscillating pressure to help prevent the accumulation of blood and/or lymphatic fluid within the limb, (e.g. functioning as a compression stocking to prevent blood clots from forming after surgery or during extended periods of patient inactivity).

In certain embodiments, a CC in accordance with the present disclosure may be sized and shaped to provide periodic and/or rhythmic chest compressions for cardiopulmonary resuscitation (“CPR”). Currently, providing chest compression for CPR fully occupies one CPR provider, and it is difficult to manually provide sufficient pressure for chest compression without breaking ribs, particularly if chest compressions are performed for an extended period. One advantage of CCs according to the present disclosure is the ability to store such CCs in places where people congregate, such as building lobbies, airport terminals, etc., and to provide simple instructions for use by an untrained or first-time user to place the uninflated CC beneath the patient and inflate and/or deflate the CC to provide chest compression for CPR.

Devices according to the embodiments of the present invention advantageously permit greater control over the pressure applied to a wound than currently-used bandages, and allow varied pressure to be applied to a wound over time, which is not possible using conventional bandages. In addition, various embodiments of the invention have features that are especially advantageous for medical applications, especially those which may be performed by emergency personnel, first responders, or battlefield personnel, including the following:
Single unibody construction containing all required elements—no removable or separately packaged parts. These elements include means for simultaneous application of multiple forces for mechanical stabilization and hemostasis, and integrated fluidic channels for wound irrigation, pharmaceutical delivery, O₂ delivery for tissue perfusion, suction (to remove blood and wound exudate, and to perform negative pressure wound therapy to increase perfusion), and control of the wound environment, all driven by on-board means (e.g., squeeze bulbs for inflation/suction by hand, or optionally, embedded CO₂ cartridges) requiring zero electrical power. All device functions are user-adjustable.
Forms a transparent sleeve upon inflation, conformal to the limb and wound, starting from a flat sheet placed under the injured limb, requiring minimal manipulation of the extremity, and avoiding further damage created by pulling an existing sleeve over wounds containing shrapnel or bone fragments. In some embodiments, the device is configured to envelop, without applying pressure to, wounds containing large fragments of foreign matter (e.g. impaling puncture wounds).
Easy to use; little to no training required; rapid application (inflation time of <1 sec for embedded CO₂ cartridges, and <60 sec for squeeze bulbs) under challenging conditions.
Incorporates a hierarchy of capabilities that can be employed as a casualty progresses through higher echelons of care until a definitive care facility is reached (e.g., rapid application of general pressure for initial point-of-injury stabilization, followed by fine-tuning of pressure to mechanically stabilize/reduce complex fractures once the casualty is in a safe area). This enables instant adaptation to the specific situation (injury type and severity, delay time until evacuation, danger of immediate environment, etc.).
Incorporates elements of definitive treatment, e.g., inflatable replacement for external stabilization devices (e.g., external fixation), negative pressure wound therapy, etc.
Constructed from flexible, biocompatible, puncture-resistant, and waterproof medical-grade elastomeric materials that selectively isolate a wound from an external environment (permits O₂ transport, but blocks H₂O vapor loss), with built-in adhesive seals—both chemical and pneumatic—working in concert to ensure attachment to the limb and easy removal without damaging the wound. Pneu-Net devices such as those described herein are also advantageously puncture resistant, and can be made more so by integration of additional puncture-resistant materials such as polyether-based thermoplastic polyurethanes.
Low profile—The proprietary Pneu-Net design allows for a lower profile when activated than current inflatable splints and devices (<1″ thick); supplied flat-pack in sealed sterile packaging.
Meets size and shape requirements (fits into a personal field medical kit, with weight, exclusive of packaging, between 250 g and 400 g).
Can be used as a standalone device without the IBC; compatible with field-available medical supplies, integrates with existing corpsman/medic practices.
Easy to manufacture in large quantities using existing commercial manufacturing methods including molding, hot embossing, RF sealing, roll to roll processing, etc.
Optionally, thin sheet magnets can be incorporated into the outermost (strain-limiting, nonextensible) layer of the CC to remove and isolate shrapnel from the wound. Magnets may also be used to form rapid connections between separate fluid lines (e.g. an external inflation line can be attached magnetically to a fluid device on the CC.

The phrase “and/or,” as used herein should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc. The term “consists essentially of means excluding other materials that contribute to function, unless otherwise defined herein. Nonetheless, such other materials may be present, collectively or individually, in trace amounts. As used in this specification, the term “substantially” or “approximately” means plus or minus 10% (e.g., by weight or by volume), and in some embodiments, plus or minus 5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

Certain embodiments of the present invention have described above. It is, however, expressly noted that the present invention is not limited to those embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

What is claimed is:

1. A conformal compression device for treatment of a wound, the device comprising pneumatic actuators and a wound dressing, wherein activation of the pneumatic actuators urges the wound dressing toward the wound.

2. The device of claim 1, wherein the pneumatic actuators include an elastomer.

3. The device of claim 1, further comprising an inlet pneumatically coupled to the pneumatic actuators, the inlet configured to connect to a source of gas.

4. The device of claim 1, wherein the device is configured to enclose at least a portion of a limb of a patient when the pneumatic actuators are activated such that the wound dressing is placed in contact with a wound on the limb of the patient.

5. The device of claim 4, further comprising a sensor configured to detect the release of a molecule that is indicative of a state of the wound.

6. The device of claim 5, further comprising a display element configured to display indicia indicative of the state of the wound as detected by the sensor.

7. The device of claim 6, further comprising at least one fluid path configured to apply irrigation or suction to the wound.

8. The device of claim 7, wherein the fluid path is in fluid communication with a port on an exterior of the device, the port being configured to provide fluid communication between the fluid path and an external fluid-handling device.

9. The device of claim 1, further comprising a directed pressure inflator on a wound-facing side of the device configured to at least partially inflate and apply direct pressure to the wound upon actuation of a membrane inflator on an exterior of the device.

10. The device of claim 1, wherein the device is substantially flat when the pneumatic actuators are not activated.

11. A method of treating a wound, the method comprising:
placing, adjacent a wound on a portion of a patient's body, a conformal compression device, the device configured to at least partially encircle the portion of the patient's body and including a wound dressing and a network of pneumatic actuators configured to urge the wound dressing toward the wound when activated; and
activating the network of pneumatic actuators.

12. The method of claim 11, wherein activating the network of pneumatic actuators comprises supplying gas to an inlet on an exterior of the device that is pneumatically coupled to the network of pneumatic actuators.

13. The method of claim 11, further comprising detecting a release of a molecule that is indicative of a state of the wound.

14. The method of claim 13, further comprising displaying indicia indicative of the state of the wound on a display element of the device.

15. The method of claim 11, further comprising applying at least one of irrigation and suction to the wound through a fluid path within the device.

16. The method of claim 15, wherein applying at least one of irrigation and suction to the wound through a fluid path within the device comprises connecting an external fluid-handling device to a port on the device.

17. The method of claim 11, further comprising inflating a directed pressure inflator on a wound-facing side of the device to apply direct pressure to the wound.

18. The method of claim 11, further comprising modulating pressurization of the network of pneumatic actuators over time.

19. The method of claim 11, further comprising pressurizing different portions of the network of pneumatic actuators to different degrees.

20. The method of claim 11, further comprising deflating the network of pneumatic actuators and substantially flattening the device.
